(19) United States
(12) Patent Application Publication
(10) Pub. No.: US 2023/0099681 A1
(43) Pub. Date: Mar. 30, 2023

(54) MEDICAL VISUALIZATION SYSTEM

(71) Applicant: SUPERSONIC IMAGINE, Aix-en-Provence (FR)

(72) Inventor: Damien LERAT, Aix-en-Provence (FR)

(73) Assignee: SUPERSONIC IMAGINE, Aix-en-Provence (FR)

(21) Appl. No.: 17/956,146

(22) Filed: Sep. 29, 2022

(30) Foreign Application Priority Data
Sep. 29, 2021 (FR) 2110274

(51) Int. Cl. A61B 8/00

(52) U.S. Cl. CPC A61B 8/54; A61B 8/467; A61B 8/465; A61B 8/464

(57) ABSTRACT

This disclosure concerns a medical visualization system to visualize medical information for the system user, configured to:
receive data representing medical information, and
receive data representing the user's position and/or the direction of the user's gaze,
the system comprising:
a visualization device which is configured to:
display an image based on the data representing the information, and
adjust the position and/or the orientation of the image displayed according to the user's position and/or the direction of the gaze.

### TECHNICAL FIELD

This invention concerns medical imaging devices. Such a system may constitute a medical examination system. More specifically, it may constitute and/or be linked to an ultrasound device.

### PRIOR ART

A medical visualization system usually includes electronic means, for example, a sensor and/or a probe (e.g., an ultrasound probe) to acquire data from a patient, and a processor to process the data acquired. It may also include a control module to pilot the system linked, in particular, to the user interface. The medical system can be used for supplying data from a medium for examination, which are displayed on a screen. With regard to medical applications, the medium is a body, for instance, a part of the patient's body (muscles, fetus, chest, liver, abdomen, etc.). In general, the probe is held by an operator or a robotic arm against the surface of an examined medium so as to acquire data on this medium.

Often the user of the medical system looks at a system screen at the same time. However, the screen is not generally directly adjacent to the medium. For instance, in a case in which a patient is examined lying on a bed or sitting in a chair, the screen is often placed on a cart (or more generally a platform) placed adjacent to the bed or the chair and, therefore, (from the point of view of the user) in a completely different direction to that of the medium. As a result, the user of the medical device is forced to adopt an uncomfortable, non-ergonomic and unnatural position while acquiring the data. The user is in particular forced to turn their head and generally to twist all or part of their torso.

In this context, international literature shows that 80% to 90% of sonographers experience pain while carrying out examinations and/or scans at one point or another in the course of their career. About 20% of them subsequently suffer a career- or life-changing injury.

There are various types of medical systems available. A medical system, for example, an ultrasound device, comes in the above-mentioned form of an ultrasound cart, generally equipped with wheels (or, more generally, on an ultrasound platform), which is configured to be moved on the floor. The cart is configured to support the relatively heavy parts of the medical system, in particular, its processing unit and its display device(s), often of a screen type.

Such systems can have a high image quality and a variety of the applications of their use (in particular, thanks to the possible use of several probes), but they lack maneuverability. Subsequently, screen devices placed on the cart or attached to it have a reduced mobility. Screen devices can be rotating and in some cases the vertical position can be adjustable. However, the relative position in relation to the user who must position themselves adequately in relation to the patient remains, in principle, substantially still the same. The user is then obliged to turn/twist their head regularly and adopt an uncomfortable and unnatural position to look at the screen during the examination of a patient.

There are also portable ultrasound devices that use, for example, a smartphone or an electronic tablet for imaging. In this case, the probe communicates with the smartphone and/or the tablet. Such systems can be easily movable. However, they generally contain only a single probe. Furthermore, they have a relatively small screen and limited self-sufficiency as their batteries are relatively small. Due to the relatively compact and light nature of the parts of a system of this type and the reduced self-sufficiency, the image quality is generally lower than that of a classic ultrasound device or still more a top of the range one. They also always have to be held in the user's hands, preventing the user from using their hands for other tasks. This may also spread hospital-acquired infections.

In addition, there are various eye-tracking techniques. For example, US2015223684A1 proposes using a camera module for eye-tracking. The rotation of a display screen can be used to trigger a change to the operational characteristics of the camera module, in particular, by using an internal IR filter of the camera module.

### INVENTION DISCLOSURE

The purpose of this disclosure is, therefore, to specify a system of medical visualization to visualize medical information which remedies the above-mentioned disadvantages. In particular, the objective is to provide a system of medical visualization which allows an optimized visualization of medical information and comfort in use. Notably, it is desirable that the medical visualization system enables the user of the system to avoid uncomfortable and/or unnatural positions while considering medical information visualized during the acquisition of data.

This disclosure concerns a medical visualization system for visualizing medical information by the system user.

The system is configured to:

receive data representing medical information, and

receive data representing the user's position and/or a direction of the user's gaze.

The system may comprise a visualization device which is configured to:

display an image based on the data representing the information, and

adjust the position and/or the orientation of the image displayed according to the position of the user and/or the direction of the gaze.

In providing such a system, it becomes possible to visualize medical information at a position and/or an orientation which suits the user of the system, indeed which allows the optimization of their working comfort, adjusting to a position considered natural for the user. In particular, the position and/or orientation of information may be adjusted so as to correspond to the direction of the user's gaze. In other words, the user is not obliged to turn their head to look at the medical information during data acquisition, such as during the examination of a patient, before the examination and/or after the examination (when the user speaks to the patient, for instance).

The adjustment may be done dynamically so that the information visualized follows the direction of the user's gaze during the user's movement (e.g., during a change of position of the user because of an operation or an examination).

For example, the position and/or orientation of the image displayed may be adjusted automatically. More generally, the system may be configured to detect a specific behavior of the user (e.g., a gesture and/or a movement, and/or taking a device in hands) and to adjust itself automatically in response. Thus, the system can allow an improvement of the ergonomics of the user's work post. The system can also improve the hygiene of the work post, because less manipulation is necessary, in particular, to manually adjust the visualization device. Furthermore, the system can ensure a reduction in the examination time because there is no need to orient and/or position the visualization device before, during, and after the examination.

The data representing the user's position may include data representing the position of the face and/or body of the user. This data can allow management of cases where the user turns their back on the patient (when they move, for example, in the scanning room, or in an intervention block).

Such data representing the position of the user's face and/or body may also be used by the system to authenticate the user. Thus, security of use can be increased, because only authorized persons having the right to use the system can do so. In the case of attempted use by an unauthorized person, the system can automatically put itself on standby, or in default security settings.

The adjustment of the position and/or orientation of the image displayed may include a rotation/swiveling of the image displayed and/or a translational shift (i.e., shifting the system from one position in the space to another position). In other words, the shift may be, for instance, a 6D movement (in six dimensions) in the space, including a tridimensional rotating shift (i.e., around three orthogonal axes), as well as a tridimensional rotating shift and tridimensional translation shift (i.e., a shift in all or part of planes x, y, and z).

The data representing the medical information may include at least one of the following:

data acquired concerning the patient, such as, for example, their administrative and personal data,

data acquired by a medical device which is manipulated by the system's user,

data acquired previously by a medical device and/or saved, and

data from the patient's file.

The file may include, for example, a medical file (including, for example, data from previous examinations) and/or administrative file of the patient. Thus, the medical information may include images, videos, and other visual or sound information, as well as text, such as the patient's name etc.

The medical device may include at least one of the following elements:

a probe or other examination device,

a sensor, and/or

an intervention device with a sensor.

The system may also include an optical sensor configured to measure the position of the user and/or of their face and/or the direction of the user's gaze.

The optical sensor may be configured to follow the position of the user and/or of the face and/or the direction of the user's gaze.

The visualization device may be configured to adjust the position and/or the orientation of the image displayed in real or quasi-real time, according to the position of the user and/or of the face and/or the direction of the gaze.

The visualization device may include a motion system including at least one motor and/or a control. The motion system may be configured to shift mechanically and/or physically the position and/or the orientation of the visualization device.

In this option, the position and/or orientation of the visualization device (e.g., a display screen device) can be shifted mechanically and/or physically. Thus, the visualization device itself can also be shifted.

The visualization device may include an optical system that comprises at least one mobile optical element. The optical system may be configured to optically project the image so that the position and/or orientation of the image displayed is adjusted.

Thus, the visualization device may also primarily maintain its position without being shifted. On the other hand, the image displayed may be shifted on the device.

For instance, it is possible for such a shift to be effected solely electronically and/or optically. If the visualization device includes, for example, a holographic screen, it is possible to orient and position the hologram so that the plane of view of the anatomy is adjusted dynamically in relation to the user. The holographic screen may be, for example, a holographic fog wall.

The mobile optical element may be configured to transmit an image in various directions. For example, the mobile optical element may swivel for this purpose. The mobile optical element may include, for example, an optical lens.

The visualization device may include a mobile display screen, optionally configured to adjust the orientation of the image displayed according to the position of the user and/or of their face.

Such mobile display screen may, for instance, be a device with a display screen, which may swivel and/or shift according to a shift in translation, notably according to the user's position and/or the direction of the user's gaze. Therefore, the display screen may be mobile.

The mobile display screen may be a touch screen and/or may include a control panel configured for allowing the user or one of their assistants or colleagues to control the system.

The visualization device may include a projector of images configured to project the image to display it, for instance, on a surface and/or display it as a hologram (e.g., in a space and/or on a fog wall), and optionally adjust the position of the image displayed according to the direction of the gaze.

The image projector may be configured to project the image facing the user (e.g., on a surface facing the user and/or displaying it as a hologram) and/or superimposed on a surface of a region of the medium, such as that for which the data is acquired.

It is, however, possible for the system to include and/or cooperate with a headset (virtual reality headset, head-mounted display, etc.) worn by the user. For example, the visualization device may display information which may also be visible for other people. The head-mounted display may also display supplementary information, which is only visible to the user who is equipped with it. For instance, the supplementary information may be superimposed and enrich and/or complete the information displayed by the visualization device.

The supplementary information may contain, for example, technical and/or confidential information, such as parts of the patient's medical file, and/or sensitive information (e.g., concerning an illness detected based on the data acquired), which is not intended for the patient or any other person possibly present.

The system may include a first mode, wherein the visualization device may be configured to adjust the position and/or orientation of the image displayed so that the image automatically follows the position of the user and/or of the face and/or the gaze of the user.

The system may include a second mode, wherein the visualization device may be configured to display the image in a set and/or user-adjustable position.

The system may be configured to receive status information representing the operational status of the medical device.

The status information may include:

information determined depending on whether the medical device is placed in a device holder or not, and/or

information determined according to at least one of the following elements:

a time of absence of shifting of the medical device (during a predefined and/or configurable period),

a status of data acquisition by the medical device, and

a status of operation according to a predefined protocol of examination and/or intervention.

The first mode may be activated and/or maintained according to the status information.

The optical sensor may be configured to detect at least a predefined gesture by the user.

The visualization device may be configured to:

shift the image displayed according to a first predefined gesture, and/or

halt or activate the first mode according to a second predefined gesture, and/or

adjust the content and/or the size of the image according to a third predefined gesture.

According to another example, if the user approaches the image displayed, the image may be automatically enlarged. This gesture may, in fact, be interpreted in such a manner that the user wishes to see the image in more detail.

According to another example, if the user moves in the room, the acquisition of data by the probe may be automatically halted. In fact, such user's behavior may be interpreted as meaning that the user no longer acquires data but that there is a change of place or patient or other modification to the examination session(s). Pointless consumption of energy can thus be automatically reduced. In addition, the cutting off of acquisition prevents the premature wear and tear of the so electromechanical elements of the probe as well as the heat engendered by the reduction in the thermal dissipation linked to the loss of contact with the medium scanned.

According to another example, the system may be configured to detect the accessories (and/or medical devices) used by the user and to react according to them. For instance, if the user grasps (or takes in the hand) a specific medical device (e.g., a probe or a biopsy needle), the system may automatically launch a specific mode.

Thus, the system may generally be configured to detect a specific behavior of the user and adjust itself automatically in response.

The visualization device may also be configured to be controllable by the user by means other than visual gestures. For example, the system may also be configured for a control by the voice and/or by a physical user interface (e.g., a control console and/or buttons for example situated on the probe and/or on the tablet or console). The medical visualization system may also include at least one of the following elements:

a medical device,

a processing unit configured to process the data acquired by the medical device and to output the data processed to the visualization device,

a second display screen configured to display at least a part of the image displayed by the visualization device and/or the user interface configured to allow the user to control the system,

a mounting device, on which the visualization device and optionally the processing unit and/or the second display screen are mounted.

The characteristics and advantages of the invention will appear upon reading the description, given solely by way of a non-exhaustive example, and made with reference to the accompanying Figures. In particular, the examples illustrated in the Figures may be combined, unless there is any significant inconsistency.

### BRIEF DESCRIPTION OF THE FIGURES

FIG. 1a schematically shows the first embodiment given by way of example of the medical visualization system according to this disclosure, in a lateral view.

FIG. 1b schematically shows the medical visualization system in FIG. 1a with optional modifications and with an adjustment of the position and/or orientation of the visualization device according to the first example.

FIG. 1c schematically shows the medical visualization system in FIG. 1a with an adjustment of the position and/or orientation of the visualization device according to the second example.

FIG. 1d schematically shows the medical visualization system in FIG. 1a with an adjustment of the position and/or orientation of the visualization device according to the third example.

FIG. 2a schematically shows the second embodiment given by way of example of the medical visualization system according to this disclosure, in a perspective view.

FIG. 2b schematically shows the medical visualization system in FIG. 2a in a perspective view of the system seen from above.

FIG. 2c schematically shows the medical visualization system in FIG. 2b with an adjustment of the position and/or orientation of the visualization device according to the fourth example.

FIG. 3a schematically shows the third embodiment given by way of example of the medical visualization system according to this disclosure, in a perspective view.

FIG. 3b schematically shows the medical visualization system in FIG. 3a in a perspective view of the system seen from above and with a projected image.

### DESCRIPTION OF EMBODIMENTS

Across the various Figures provided for illustrative purposes, the same numerical references denote the same or similar elements. The different embodiments found in the Figures may be combined unless there is any significant inconsistency.

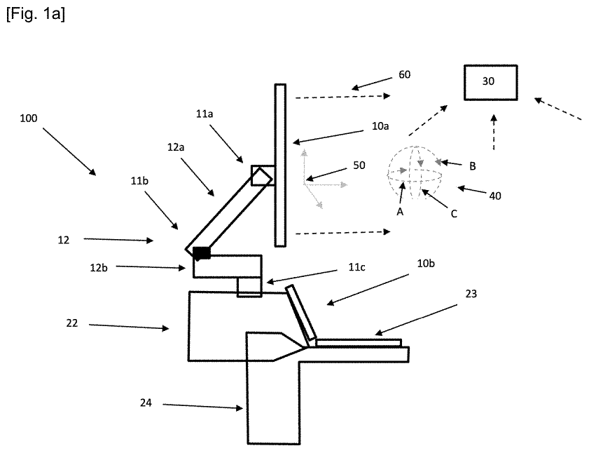

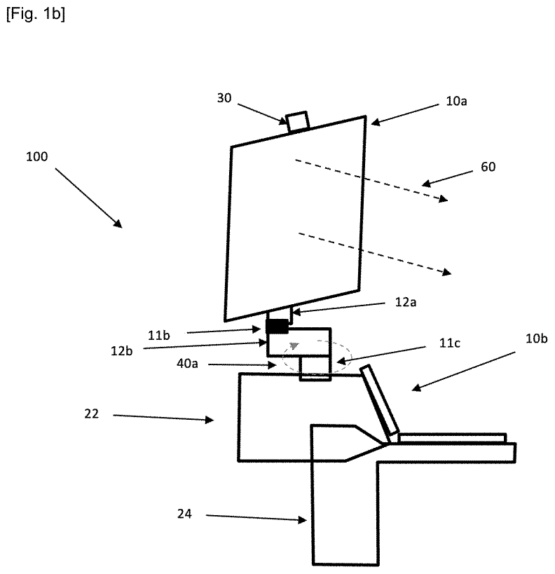

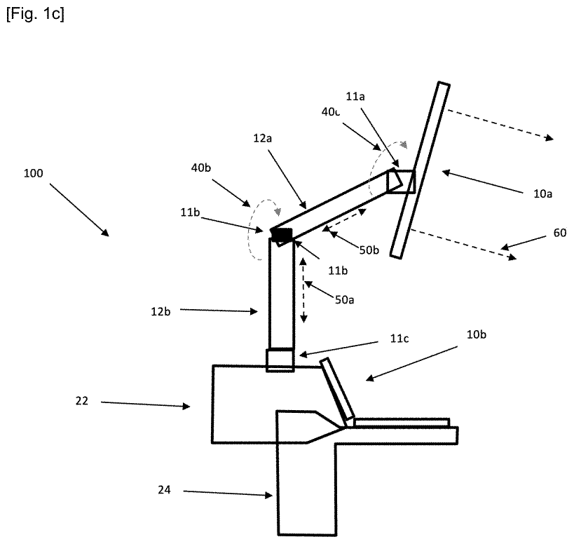

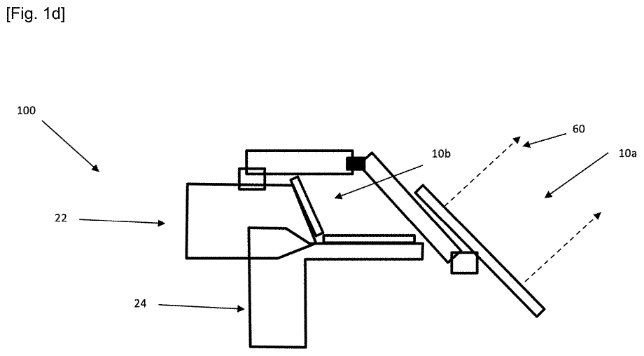

FIG. 1a schematically shows the first embodiment given by way of example of the medical visualization system according to this disclosure, in a lateral view.

The medical visualization system 100 may be configured to visualize medical information for the system user.

The system 100 may be a system of medical imaging (e.g., for an examination of a patient and/or for veterinary use or for any other imaging application), in particular, an ultrasound system. Other examples of such systems include an optical imaging system, an x-ray system, a computer tomography system, a mammography system, among others.

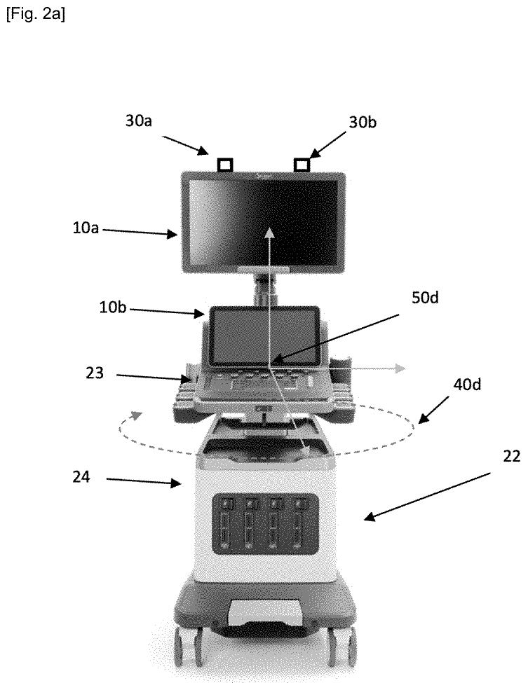

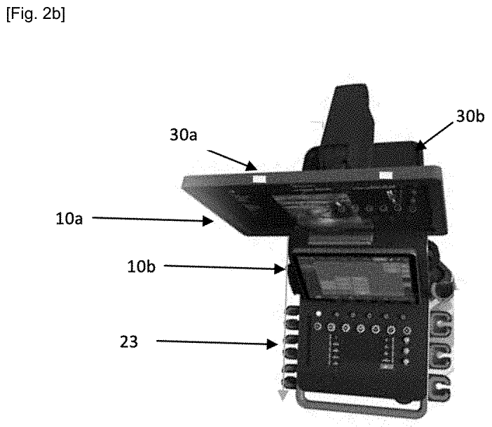

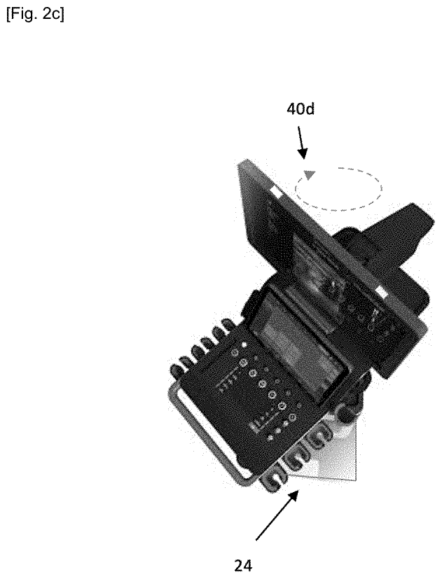

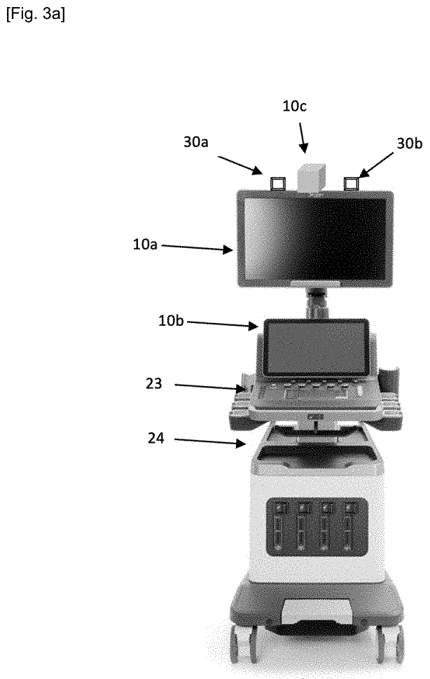

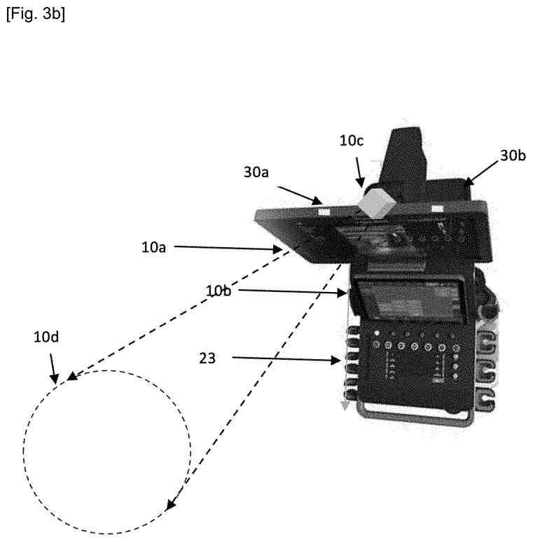

The system is configured to receive data representing the medical information (e.g., acquired with the aid of a probe, not illustrated in FIG. 1a), and to receive data representing the user's position and/or the direction of the user's gaze (e.g., by the sensor 30 in FIG. 1a or the sensors 30a, 30b in FIG. 2a). The system includes at least one visualization device 10a, 10b (cf. for example FIG. 1a), and/or 10c (cf. for example FIG. 3a) which is configured to display an image based on the data representing the information, and adjust the position and/or orientation of the image displayed according to the user's position and/or of the direction of the gaze.

The system 100 may also include a master station 22 including, for instance, electronic and/or computing resources. The examples illustrated here include a processing unit and/or one or several storage areas. The said computing resources may be used to process the examination data. It is, however, also possible for these resources to be remotely deployed (e.g., on a work station, in the cloud, etc.).

The master station 22 may be connected to or may include one or several sensors and/or probes (not illustrated in FIG. 1a). The said sensors and/or probes may be configured to acquire the examination data from a person examined and/or practice interventions.

The visualization device may communicate with the master station 22 via a cable, but also alternatively via a wireless interface, for example, by using protocols such as WIFI®, BLUETOOTH® or others. The master station may also supply a device with a touch screen in electrical energy, which may also be realized alternatively via a connector or wirelessly.

The master station may be placed on a foot or on a base 24 (not entirely represented in FIG. 1a). The foot 24 may also include the master station 22 (cf. for example FIG. 2a). In particular, the mounting device or foot 24 may have a height adapted or adaptable to the user's size, that is such that the visualization device 10a is at an appropriate level for the user and/or adaptable to the type of examination performed.

The system, in particular the visualization device, may include a first display screen 10a.

The system, in particular the visualization device, may also include a second display screen 10b. At least one of the first and second display screens 10a, 10b may be a touch screen.

The system may also include the user interface and/or a control panel 23 configured to allow the user to control the system. The control panel may, for example, include one or several buttons, sliding controllers, and/or a touch pad.

As already mentioned, the system may include and/or may be configured to cooperate with a medical device (not represented in FIG. 1a), for instance, a probe or another examination device, a sensor, and/or an intervention device with a sensor.

The probe may for example be an ultrasound probe. However, the probe may also be configured to use technologies other than the ultrasound acquisition technique. For example, it may be or comprise other types of sensors or combined data acquisition techniques. In one example, the probe may comprise one or more sensors and/or optical transducers and/or lasers. It is also possible for the probe to be configured for optical/acoustic data acquisition. As a result, the processing unit may also be configured to process the types of data other than ultrasound data, so as to generate information for the purposes of display. Moreover, besides the probe, the system may also be configured to cooperate with other types of medical devices, for example, a marker and one or more surgical devices (e.g., a device configured to extract material from the medium, such as, for instance, a biopsy needle or any surgical instrument used in combination with the device). As a result, the system 100 may be configured to comprise or be used with any combination of these probes, sensors, and medical devices.

The system may also comprise or be configured to cooperate with an optical sensor 30 configured to localize and/or measure the position of the user and/or of their face and/or the direction of the user's gaze. However, as shown in the example in FIG. 1a, the sensor may also be an external device in relation to the system 100. For instance, the optical sensor 30 may be placed in a room or environment where the system is used, so that the sensor 30 can detect the user's position and/or the direction of the user's gaze.

In addition, at least one of the first and second display screens 10a, 10b may be configured so that its position and/or its orientation are automatically adjusted in response to the user's position or the direction of the user's gaze.

In particular, the adjustment may include a transversal shift in at least one axis direction in a tridimensional Cartesian coordinates system 50 (i.e., in at least one of the directions x, y, z) and/or a rotation shift in at least one direction of rotation in a tridimensional polar coordinates system 40 (i.e., in at least one of the axes A, B, C). Thus, the shift may include the shift components between 1D (uni-dimensional) and 6D (six dimensions). A 6D shift (six dimensions) may include a tridimensional rotation shift and a tridimensional translation shift (i.e., a shift in planes x, y, and z).

In the example illustrated in FIG. 1a, at least the position and/or the orientation of the first display screen 10a is adjustable. The position represented in FIG. 1a may also be designed as a position by default and/or of parking of the first display screen 10a. However, some corresponding elements and/or setting functions (i.e., of adjustment) may also be present for the second display screen 10b or any other screen.

The first display screen 10a may include a motion system including at least one motor and/or a servo-motor. The motion system may be configured to shift mechanically and/or physically the position and/or the orientation of the first display screen 10a. Thus, the first display screen 10a may itself be shifted.

In particular, the first display screen 10a may be mounted on a mobile support device 12. The mobile support device 12 may include at least one arm 12a, 12b. Also, the mobile support device 12 may include at least one swiveling hinge (or joint) 11a, 11b, 11c. This swiveling hinge 11a, 11b, 11c may include a hinge 11a between an arm 12a and the display screen, a hinge 11b between a first arm 12a and a second arm 12b, and/or a hinge 11c between an arm 12b and the mounting device 24 or any other element of the system (e.g., the master station 22). The hinge(s) may be rotating around one or several axes. At least one of the arms may be extensible and retractable, so that its length can be adjusted according to use and mode. The swiveling shift of the hinges and the translation shift of an extensible arm may be activated by a motor and/or servo-motor.

Thus, thanks to these swiveling and translation shifts, the first display screen 10a can be shifted in translation and/or in rotation. The mobile support device 12 may for example be configured to allow any orientation of the first display screen 10a. More particularly, the mobile support device 12 may enable a rotation of the display screen 10a (seen from the display direction 60) around a first axis A (i.e., a vertical axis) and/or a second axis B (that is a horizontal axis), the two axes being orthogonal Also, the mobile support device 12 may for example be configured to allow any translation shift in the possible band of the mobile support device 12 fully deployed.

The first display screen may be configured to adjust the position and/or the orientation in real or quasi-real time according to the position and/or of the face and/or the direction of the gaze of the user. For example, the first display screen 10a may be configured to adjust the position and/or orientation so that the screen automatically follows the position of the user and/or of the face and/or the gaze of the user. Thus, the user can move or change position (e.g., from standing to sitting position), without having to adjust the position of the screen while being able to continue to consult the information displayed. The principal direction of display 60 of the first display screen 10a may then follow the position and/or direction of the user's gaze, so that the user can benefit from seeing the information displayed. Even if a shift in translation of the first display screen 10a may be limited because of the maximum length of the mobile support device 12 fully deployed, the first display screen 10a may be turned around the first axis A (i.e., a vertical axis) and/or the second axis B (i.e., a horizontal axis in the display plane) to follow the position of the user and/or the position of the head and/or the face of the user.

The said automatic adjustment may be effected by the system at least in a first mode.

The system may also include a second mode in which the first display screen 10a may be configured to remain in a position set and/or adjusted by the user. Thus, the user cannot be distracted by a shift of the first display screen 10a, when the first mode is not desired by the user.

For example, the user can activate and/or deactivate the first mode manually, by a vocal command or any other system of man-machine interface. Alternatively, or in addition, the first mode may be activated and/or maintained on the basis of status information representing the status of functioning of the medical device. The status information may include, for example, information determined according to the fact that the medical device is placed in a mounting device, and/or information determined according to at least one of the following elements: a time of absence of shifting the medical device (over a predefined and/or configurable period), and/or of a status of data acquisition by the medical device, and/or a status of functioning according to a predefined protocol of examination and/or of intervention.

Also, the optical sensor 30 can detect at least one predefined gesture by the user. In response, the first display screen 10a may shift according to a first detected predefined gesture, and/or halt or activate the first mode according to a second detected predefined gesture, and/or adjust the content and/or the size of an image displayed according to a third predefined gesture of the first display screen 10a.

The shifting of the first display screen 10a may also be controlled by the user by means of gestures other than visual ones. For instance, the system may also be configured to be commanded by the voice and/or by a physical user interface 23, a remote control, etc.

Furthermore, the shifting may be controlled by sensors of different types (e.g., the optical sensor 30 and/or one or several distance sensors set on the first display screen 10a), which recognize an object in the environment of the system (e.g., a bed, a seat, or a patient, as well as the walls or the ceiling of the room). Thus, the shifting may be limited so as to stop the first display screen 10a touching such objects (e.g., a patient).

FIG. 1b schematically represents the medical visualization system in FIG. 1a with optional modifications and with a position and/or adjusted orientation of the visualization device according to a first example.

The embodiment according to the example in FIG. 1b may correspond to that of FIG. 1a. In particular, the embodiment according to the example in FIG. 1b may comprise the same elements and/or functions.

However, in the illustrated example, the sensor 30 may be attached to the system 100 and/or form part thereof. For instance, the sensor may be set on the first display screen 10a, for example, on its higher part.

In the example provided in FIG. 1b, the mobile support device 12 may be configured to enable a rotation of the first display screen 10a (seen from the direction of display 60) at least (or solely) around a vertical axis. This example may constitute an elementary embodiment of this disclosure. In the illustrated adjustment position of the first display screen 10a (in relation to the position of the example in FIG. 1a), the first display screen 10a has been turned in a clockwise direction 40a, seen from above. In particular, the first display screen 10a can be turned around the hinge 11c. However, it can also turn around any other hinge, for example, the hinge 11b or the hinge 11a.

The first display screen 10a can freely rotate at 360°. Alternatively, the first display screen 10a can swivel at 180° (or less) in both directions of rotation, starting from the default position represented in FIG. 1a. This alternative may be used, for instance, when the first display screen 10a is linked by a cable to the master station 22 which does not allow one or several rotations of 360°.

FIG. 1c schematically represents the medical visualization system in FIG. 1a with a position and/or adjusted orientation of the display device according to a second example.

The embodiment according to the example represented in FIG. 1c may correspond to that in FIG. 1a or 1b. In particular, the embodiment according to the example in FIG. 1c may comprise the same elements and/or functions. In particular, the embodiment according to the example in FIG. 1c may comprise the same functions of rotation as that in FIG. 1b.

Furthermore, the arms 12a and 12b may be extensible and retractable. In the illustrated adjustment position of the first display screen 10a (in relation to the position of the example in FIG. 1a), the two arms 12a, 12b can be extended so that the first display screen 10a can be shifted in translation towards an extended position, in the example illustrated towards a raised position and protruding horizontally. The hinges 11b and 11c can also be turned, so that the arms 12a and 12b unfold. In addition, hinge 11a can be turned, so that the first display screen 10a is slightly inclined downwards.

FIG. 1d schematically represents the medical visualization system in FIG. 1a with an adjusted position and/or orientation of the display device according to a third example.

The embodiment according to the example provided in FIG. 1d may correspond to that in FIG. 1a, 1b, or 1c. In particular, the embodiment according to the example in FIG. 1c may comprise the same elements and/or functions. More particularly, the embodiment according to the example in FIG. 1d may comprise the same functions of rotation and extension as that in FIG. 1c. Also, in the illustrated adjustment position of the first display screen 10a, the arms 12a, 12b can be extended in the same way as in FIG. 1c. However, in relation to the position of the example in FIG. 1c, the hinges 11b and 11c can be turned, so that the arms 12a and 12b extend towards a lower position which can still be protruding in relation to the mounting device 24 in a horizontal direction (for example towards the user situated to the right of the system in FIG. 1d). Also, hinge 11a can be turned, so that the first display screen 10a is slightly inclined upwards.

The illustrated adjustment of the position and orientation of the first display screen 10a may follow the direction of the user's gaze. For instance, the user may have a slightly distant position in relation to the system, for example, because of a patient positioned between the user and the system. The patient may be positioned for example on a bed or on a seat.

For instance, in the position indicated in FIG. 1c, the user may start an examination procedure. Thus, the user can look slightly upwards, towards a point situated above the patient. For example, the user can make a selection from a menu displayed on the first display screen 10a, for instance, via a gesture or another type of command, as described above. Then, in the position specified in FIG. 1d, the user can acquire data starting from a medium of the patient by using a probe. Thus, the user can look slightly downwards in the direction of a point of the surface of the patient, where the probe is located. Consequently, the first display screen 10a can be shifted from the position and orientation indicated in FIG. 1c to those in FIG. 1d. Subsequently, the user can monitor any information provided on the first display screen 10a in the context of the acquisition of data. At the same time, the direction of the user's gaze can remain substantially focused on the medium point examined, such as on a part of the patient's body.

FIG. 2a schematically represents the second example of embodiment of the medical visualization system according to this disclosure, in a perspective view. The embodiment according to the example represented in FIG. 2a may correspond to that of any of the other Figures. In particular, the embodiment according to the example provided in FIG. 2a may comprise the same elements and/or functions.

However, in the illustrated example, the mounting device 24 may be configured so that an upper end of the mounting device 24 rotates around a vertical axis, for example in a direction 40d and/or in its opposed direction. Optionally, the foot or base 24 may comprise the master station 22. Hence, the combination of the higher parts of the system 100 (comprising for example: the master station 22 (as an option, if it is not located in the foot), the user interface 23 and the first and second display screens 10a, 10b) may be rotating. In another option, the mounting device 24 may be configured to allow a shift in translation of these higher parts in at least one axis direction of a tridimensional Cartesian coordinates system 50d.

Furthermore, as illustrated in the example provided in FIG. 2a, the system may include at least two optical sensors 30a, 30b, for example, set on the first display screen 10a, to ensure a better localization of the user in a tridimensional space. In addition, the two optical sensors 30a, 30b may present principally the same characteristics and/or functions as the optical sensor 30 described above.

FIG. 2b schematically shows the medical visualization system in FIG. 2a in a perspective view of the system seen from above. FIG. 2b schematically shows the medical visualization system in FIG. 2a with an adjusted position and/or orientation of the display device according to a fourth example.

As demonstrated in FIG. 2c, the combination of all parts of the system 100 (including for example: the master station 22 (as an option, if it is not located in the foot), the user interface 23 and the first and second display screens 10a, 10b) may be oriented in relation to the mounting device 24 in a direction 40d and/or in its opposed direction. Thus, the system may automatically orient all its higher parts towards the position of the user. Hence, the user can move around the system 100 while continuing to see the information on the first display screen 10a, the second display screen 10b. Also, the user can make any command with aid of the control panel 23.

FIG. 3a schematically represents the third example of embodiment of a medical visualization system according to this disclosure, in a perspective view. The embodiment according to the example represented in FIG. 3a may correspond to that of any of the preceding Figures. In particular, the embodiment according to the example provided in FIG. 3a may comprise the same elements and/or functions.

However, in the illustrated example, the system, in particular the visualization device, may include an image projector 10c.

In one option, the image projector 10c can mechanically adjust its position and/or its orientation according to the position and/or the direction of the user's gaze (as indicated in the example indicated in FIGS. 3a and 3b).

In addition, or alternatively, the image projector 10c may include an optical system comprising at least one mobile optical element. The optical system may be configured to optically and/or electronically control a direction of projection, so that the position and/or orientation of the image displayed can be adjusted, automatically or by the user.

The system 100 may include a combination of the image projector 10c and the first and/or second display screen 10a, 10b. Alternatively, the image projector 10c may also replace or complete at least one of the first and second display screens 10a, 10b.

FIG. 3b schematically represents the medical visualization system in FIG. 3a in a perspective view of the system seen from above and with a projected image. The image projector 10c may be configured so that the position and/or orientation of a projected image 10d can be adjusted according to the position and/or direction of gaze of the user and the position of the walls of the room. The adjustment may follow the same principles, as described above in the context of the first display screen 10a. For example, the image projector 10c may project the image 10d so that it faces the user.

In one example, the image projector 10c may project the image 10d on a surface facing the user and/or project it as a 3D hologram in the space and/or as a 2D hologram on a virtual wall (e.g., a fog wall). In addition, the image projector 10c may also project the image 10d so that it is superimposed on a surface of a region of the support from which the data is acquired (e.g., on the patient).

Moreover, the system may detect the geometry of the room by using the sensors 30a, 30b, in which the system is situated. For example, the system may detect the lines formed by the transitions between the walls and the ceiling/floor, and/or between the walls to deduce from this the orientation and position of the walls in relation to the image projector 10c. Thus, the system may take into account such geometrical information, during the projection, for example, to project an image on a wall of the room and/or project a hologram into the room.

All of these embodiments and other examples as described above are given solely by way of a non-limiting example and may be combined and/or modified within the scope of the following claims.

A reference to a patent document or any other element identified as being prior art may not be considered to be an admission that the document or the other element was known or that the information it contains was part of general common knowledge at the priority date of any of the claims.

1. A medical visualization system to visualize medical information for the system user, configured to:
receive data representing medical information, and
receive data representing the at least one of user's position and a direction of the user's gaze, the system comprising:
a visualization device which is configured to:
display an image based on the data representing the information, and
adjust at least one of a position and an orientation of the image displayed according to the at least one of user's position and the direction of the user's gaze.

2. A medical visualization system according to claim 1, wherein the data represents medical information including at least one of:
data acquired relating to a patient,
data acquired by a medical device which is manipulated by the system user,
data at least one of acquired previously by a medical device and saved, and
data from a patient file.

3. A medical visualization system according to claim 2, wherein the medical device comprises at least one of:
one of a probe and an examination device,
a sensor, and
an intervention device with a sensor.

4. A medical visualization system according to claim 1, wherein the system also comprises:
an optical sensor configured to measure at least one of the user's position and the direction of the user's gaze.

5. A medical visualization system according to claim 4, wherein at least one of:
the optical sensor is configured to follow at least one of the user's position and the direction of the user's gaze, and
the visualization device is configured to adjust at least one of the position and the orientation of the image displayed in real time, according to the at least one of user's position and the direction of the gaze.

6. A medical visualization system according to claim 1, wherein the visualization device includes a motion system, the motion system comprising at least one of motor and a servo control, the motion system being configured to mechanically shift the at least one of position and the orientation of the visualization device.

7. A medical visualization system according to claim 1, wherein the visualization device comprises an optical system, the optical system comprising at least one mobile optical element, the optical system being configured to optically project the image, so that the at least one of position and orientation of the image is adjusted.

8. A medical visualization system according to claim 1, wherein the visualization device at least one of includes a mobile display screen, and is configured to adjust the orientation of the image displayed according to the user's position.

9. A medical visualization system according to claim 8, wherein the mobile display screen at least one of is a touch screen and includes a control panel configured to enable the user to command the system.

10. A medical visualization system according to claim 1, wherein the visualization device includes an image projector configured to at least one of:
project the image to at least one of display the image on a surface and display the image as hologram, and
adjust the position of the image displayed according to the direction of the user's gaze.

11. A medical visualization system according to claim 10, wherein the image projector is configured to project the image at least one of facing the user and superimposed on a surface of a region of the medium for which the data is acquired.

12. A medical visualization system according to claim 1, wherein the system includes a first mode, in which the visualization device is configured to adjust at least one of the position and the orientation of the image displayed so that the image automatically follows the at least one of user's position and gaze.

13. A medical visualization system according to claim 12, wherein the system includes a second mode in which the visualization device is configured to display the image in at least one of a set and a user-adjustable position.

14. A medical visualization system according to claim 1, wherein the system is configured to receive status information representing operational status information of the medical device.

15. A medical visualization system according to claim 14, wherein the operational status information includes at least one of:
information determined depending on whether the medical device is placed in a device holder or is not placed in the device holder, and
information determined according to at least one of:
a time of absence of shifting the medical device,
a status of acquisition of data by the medical device, and
a status of operation according to a predefined protocol of at least one of examination and intervention.

16. A medical visualization system according to claim 12, wherein the first mode is at least one of activated and maintained according to the operational status information.

17. A medical visualization system according to claim 12, wherein at least one of an optical sensor is configured to detect at least one predefined gesture by the user, and
the visualization device is configured to at least one of:
shift the image displayed according to a first predefined gesture,
halt or activate the first mode according to a second predefined gesture, and
adjust at least one of the content and the size of the image according to a third predefined gesture.

18. A medical visualization system according to claim 1, wherein the medical visualization system further includes at least one of:
a medical device,
a processing unit configured to process data acquired by the medical device and to output the data processed to the visualization device,
a second display screen configured to display at least a part of the image displayed by at least one of the visualization device and the user interface configured to allow the user to control the system, and
a mounting device, on which at least one of the visualization device, the processing unit and the second display screen are mounted.
